(19) United States
(12) Patent Application Publication
(10) Pub. No.: US 2023/0134377 A1
(43) Pub. Date: May 4, 2023

(54) INSECT-REPELLENT PERSONAL-CARE COMPOSITION

(71) Applicant: One-Derings LLC, Clarksville, OH (US)

(72) Inventors: Amy Farr, Clarksville, OH (US); Kim Benz, Clarksville, OH (US); Karen Kreutz, Cincinnati, OH (US); Dean DuVal, Sioux Falls, ND (US)

(21) Appl. No.: 17/977,467

(22) Filed: Oct. 31, 2022

Related U.S. Application Data
(60) Provisional application No. 63/274,010, filed on Oct. 31, 2021.

(51) Int. Cl. A01N 65/44; A01N 25/02; A01N 65/06; A01N 65/22; A61K 8/27; A61K 8/29; A61K 8/9789; A61K 8/9794; A61K 8/67; A61Q 17/04; A61Q 17/02

(52) U.S. Cl. CPC A01N 65/44; A01N 25/02; A01N 65/06; A01N 65/22; A61K 8/27; A61K 8/29; A61K 8/9789; A61K 8/9794; A61K 8/678; A61Q 17/04; A61Q 17/02

(57) ABSTRACT

An insect repellent composition comprising: a solvent system containing medium-chain triglycerides and a co-solvent. The co-solvent comprising an alkyl chain comprising between eight and fourteen carbon atoms and wherein the co-solvent is selected from a group consisting of an alkyl methyl ester, an alkyl ethyl ester, an alkyl isopropyl ester, an alkyl carboxylic acid, an alkyl methyl ketone, an alkyl ethyl ketone, an alkyl alcohol, an alkyl isethionate, and combinations thereof.

### CROSS REFERENCE TO RELATED APPLICATIONS

This application claims the benefit of U.S. provisional patent application 63/274,010, filed Oct. 31, 2021; the disclosure of which is hereby incorporated by reference in its entirety.

### TECHNICAL FIELD

The present disclosure relates to an insect-repellent personal-care composition. Specifically, the composition: is at least partially natural-derived, comprises one or more natural actives, has good phase stability, and has excellent long-term lab efficacy and consumer effectiveness in use against biting hematophagous arthropods.

### BACKGROUND

The market of natural insect repellents is currently limited to blends of essential oils or their derivatives as the ‘actives’. A popular one is Oil of Lemon Eucalyptus or its active ingredient p-Menthane-3,8-diol (PMD), which can be extracted or synthesized. Another potential natural repellent is pyrethrum, which can be extracted from marigolds, but it appears all current products on the market are using a synthetic version of the active, and the natural version is not being utilized. The tradeoff(s) users make consist of a shorter duration of effectiveness due to the active(s)' volatility and/or a reduced efficacy in terms of the range of insects repelled and/or suboptimal skin use aesthetics. Additionally, product labels can contain warnings such as: ‘do not use on children under 3’ and/or ‘flammable’ and/or ‘do not apply over cuts, wounds or irritated skin’ and/or ‘apply only on exposed skin or clothing’ and/or limitations in the number of times it can be reapplied and/or ‘after returning from outdoors, wash product from all exposed skin’.

The identified art utilizes one or two types of insect repellent actives and tries to compound the active(s) in a matrix without fully recognizing and utilizing the affect the matrix has on the volatility of the active ingredient to provide an extended efficacy insect repellent. Further, the actives are deployed in a manner to only deliver a short and/ mid-term efficacy. None of the references identified were compounded to provide an extended efficacy in a single, cost-effective composition that is simultaneously personal-care-composition compatible.

Relating to fatty acid and essential oil actives, W. A. Skinner et al. first demonstrated and disclosed that skin lipids could provide at least a mild mosquito repellency effect back in the 1960s and 1970s.

U.S. Pat. No. 5,594,029 to Bencsits disclosed the use of “first runnings” coconut fatty acids combined with a variety of vegetable oils as a mosquito repellent, filed in 1995. Bencsits also disclosed other people's work in this area in the 1980s. “First runnings” would commonly refer to the first distillation pass of the fractionated fatty acids from the associated oil. In this case, it would likely be C6, C8, and C10 fatty acids.

Reifenrath et al.'s work built upon the insights of Skinner and Bencsits and filed a series of patents beginning in 1997. U.S. Pat. Nos. 9,545,100, 6,444,216, and 6,306,415 as well Canadian Patent No. 2,295,988 teach the use of lower, intermediate, and higher volatility organic compounds based on carboxylic acids and their alcohol, ketone, and lactone homologs wherein their carbon backbones contained between 8 and 12 carbon atoms. They focus on compositions with a 1:1:1 ratio of fatty acids and teach the use of a dermatologically compatible carrier, such as a volatile silicone, petrolatum, lanolin, and various others.

U.S. Patent Publication No. 2019/0141999 to Zhu and Cermak further built upon Reifenrath et al.'s work utilizing C8 to C12 fatty acids, including odd-chain acids as their insect repellent. They indicate that the fatty acids may be branched or straight chained, methyl, ethyl, propyl, isopropyl, or 2-ethyl hexyl esters of said fatty acids and their salts. They utilize the light and mid-cut fatty acids based on relative volatility and ability to provide a broad insect repellent efficacy over time based on their studies. They also claim an optional carrier but used an emulsion in the compositions tested.

German Patent No. 19,925,838 to Deutal et al. disclose the use of C12 fatty acid for the repellency or removal of ticks from skin. Their product was compounded in an alcohol matrix. They did disclose an aqueous solution containing —10% active with about 1% Sodium Dodecyl Sulfate (SDS). They also disclose that at high concentrations it can repel mosquitos but in low concentrations, it can be an attractant. It was also described as an attractant for mites. They also disclose potential forms to be cosmetic pastes, ointments, creams, gels, skin oils, sticks, sprays, lotions, solutions, tinctures, emulsions, powders, microcapsules, aerosols, or shampoos, as well as granules, powders, or sprays in slow-release compositions. However, none of these forms were enabled. The combination of alcohol and SDS could cause drying and irritation of the skin and would be negatively perceived as harsh by consumers.

European Patent Publication No. 3,771,335 filed by Athenion A G discloses an insect repellent consisting of margosa oil (neem oil), lauric acid, and a spreading agent. However, the publication also acknowledged that margosa oil is a yellow-brown liquid with a noticeable unpleasant odor and oily viscous consistency requiring the use of a large amount of spreading agent. Further, recently there have been reports of acute onset of severe metabolic acidosis, hepatic and multi-organ failure and death following ingestion of margosa oil, particularly in young children. The acknowledged smell, appearance, and feel negatives, plus potential health effects of margosa oil would make it highly undesirable as a repellent active in a skin-care type of consumer product.

U.S. Patent Publication No. 2021/0144995 to Vidal et al. disclose non-aqueous insect repellents consisting of lauric acid and a solvent, including as solvent examples alcohols, ketones, mineral oils, siloxanes, silicones, terpenes, and other potential solvents. The publication remained silent regarding; the negative skin-drying or pore-plugging effects associated with using non-aqueous solvents such as alcohols, silicones, polyethylene or polypropylene glycols, petrolatum, or mineral oils, the negative consumer issues associated with petroleum-based or synthetic solvents, or the solubility issues associated with incorporating a single chain-length fatty acid in a solvent also containing terpenes or essential oils.

There are additional teachings that point away from the present disclosure or have limitations with being able to be compounded in skin care like products. Each has ‘issues’ with regard to desirability of using on skin—whether it be the carrier and skin aesthetics. In addition, Riefenrath and Zhu explicitly include the potential use of C9, which is highly undesirable for use on humans given that it is classified as an herbicide. Riefenrath additionally discloses carriers which are suboptimal for application to human skin and do not teach solutions that in essence are for cosmetic skin care products.

Another issue with functionality with regards to Zhu and Cermak is that the stated salts of the fatty acids would be at the interface of the aqueous and organic phase. Being ion pairs with an inorganic anion also means that they have essentially no volatility and would therefore be rendered inactive in that matrix. To note, they tested coconut oil using an in-lab repellency test and found no efficacy for coconut oil so in essence, teaching away from the use of triglycerides high in C12 fatty acid or other short to mid-chained fatty acids as an active ingredient.

U.S. Patent Publication No. 2018/0035674 to Schmidt et al., teaches the combination of C8 and C10 fatty acids, in combination with geraniol and peppermint essential oils in a bar or liquid soap context. The fatty acids are either partially or completely saponified (made into fatty acid salts or soaps) using alkali metal hydroxides such as sodium or potassium hydroxide. The use of a sodium salt would lead to a solid soap while use of a potassium salt would lead to the formation of a liquid soap. Both matrices would be emulsions containing water and oil phases. Schmidt also discloses the use of triglycerides made from C8 and C10 fatty acids, also known as Medium-Chain Triglycerides (MCTs). Regardless of the starting material, i.e. fatty acids or triglycerides, both species would produce non-volatile C8 and C10 fatty acid salts or “soaps” in the matrix as described.

U.S. Pat. No. 7,575,765 to Hughes teaches the use of MCTs as a shelf-stable carrier for essential oils due to the carrier's saturated fatty acids. The essential oils used include cedarwood, citronella and lemongrass oils up to about 15% in a matrix of “fractionated coconut oil.” Fractionated coconut oil being another term used to describe MCTs. However, the patent teaches away from using other actives such as light or mid-cut fatty acids. The most obvious chemical difference is that no one has combined a mixture of essential oils, mid-cut fatty acids, medium-chain triglycerides, and a natural-sourced co-solvent in a homogeneous matrix for use as an insect repellent. Further, the art teaches away from the combination by either using emulsions, soaps, or completely non-polar matrices which would significantly affect the volatility and/or availability of some or all of the active ingredients.

The prior art which recognizes the effect of volatility on efficacy (Reifenrath and Zhu), utilizes the volatility differences between chain-lengths or polar groups of volatile fatty acids or similar actives to prolong the efficacy of the actives. They do not recognize that low volatile or nonvolatile actives (including those derived from coconut oil) can also be used to repel insects and arthropods in a skin care product. Zhu, in-fact, demonstrates that coconut oil does not have any efficacy based on the in-lab test methodology.

Unfortunately, their in-lab testing does not accurately reflect what occurs on the skin surface, and therefore teaches away from an effective way of prolonging the efficacy of the fatty acid insect repellent. They do not recognize that there is another means of delaying volatility of their primary fatty acid actives: derivatization. The esterases from microbes naturally found on human skin will hydrolyze the mid-chain triglyceride (MCTs) over time to diglycerides, then monoglycerides, and finally to C8 and C10 fatty acids. However, Unilever (Meyer et al.) proved that esterases on the skin would hydrolyze ester linkages of their PMD esterified active, thereby resulting in a delayed release profile of a normally volatile active.

Another differentiation from the prior art is the impact of usage on human skin. Prior art used C9 fatty acid, which is an herbicide, in a silicone or mineral oil carrier, which are inferior for moisturization of skin and can trap dirt and clog pores. Products using a combination of C9 and mineral oil or silicone have instructions that if you get the product on human skin, it should be washed off immediately.

No one has developed an effective, safe, natural, and long-lasting insect repellent that consumers are willing to leave on skin over a long period of time. Either the consumers are concerned about the safety of the active, such as with DEET compositions, or the so-called “natural” or “safe” compositions (such as citronella-containing compositions) are just not that efficacious.

The challenges addressed by the present disclosure were to improve 1) cost efficiency, 2) repellency efficacy, 3) human safety profile, and 4) pleasant aesthetics of the actives and carrier. The composition needs to have pleasant-enough aesthetics that consumers will be happy to put it on and leave it on their skin.

The initial approach was simply to go to higher levels of C12 fatty acid and lower levels of C10 fatty acid and eliminate C8 fatty acid, as the C12 fatty acid is the dominate chain length in coconut oil (feed stock), so it has a significantly better cost profile in the open market. C12 fatty acid has a strong performance profile based on technical tests on mosquitos, biting flies, and ticks, so upping the total fatty acid concentration would theoretically yield higher repellency efficiency, and C12 fatty acid has a very strong safety profile, so this change should have addressed all three goals in one simple composition change. Unfortunately, this change created two unexpected issues.

The first issue was one of phase stability, i.e., with higher levels of C12 fatty acid, the fatty acid precipitates out of the MCT matrix when essential oils are also present, leaving just the essential oils and the MCT carrier in solution. This was completely unexpected because C12 fatty acid is completely soluble in MCTs and so are the essential oils that were present in the composition.

The second issue was that C12 fatty acid appear to behave as a close-proximity or contact repellent, wherein the mosquito, biting fly, tick, or other repellent will land but not bite, unless it finds an area of skin that has not been treated with the product.

Accordingly, in light of these two unexpected issues, there is a need for a composition with long-term efficacy in a phase-stable composition containing high levels of C12 fatty acid, zero or low levels of both C8 and C9 fatty acids, an additional spatial repellent at efficacious levels, and a co-solvent or carrier capable of solubilizing a mixture of essential oils (“EO”) and C12 fatty acid in a MCT solvent matrix. Further, there exists a need for a true natural-active based insect-repellent composition that is not only safe to use on skin, but also skin beneficial. There is a desire to create such a safe composition in a cost-effective manner. There is a desire for all (or at least a significant majority of) ingredients in the product to be natural or natural-derived. There is a desire for this natural product to be beneficial to the skin in terms of moisture-loss prevention and skin-softening benefits. There is a desire for this natural-active insect repellent composition to be more efficacious against mosquitos, biting flies, and ticks in comparison to other natural products and synthetic products containing DEET, IR3535, or Picaridin. Consumers also have a long-felt but unmet need for a natural insect repellent that is as effective (if not more effective), longer lasting, and safer on skin than synthetic insect repellents currently on the market.

### SUMMARY

One or more embodiments of the present disclosure relate to an insect repellent composition comprising: medium-chain triglycerides and a co-solvent, wherein the co-solvent comprises an alkyl chain comprising between eight and fourteen carbon atoms.

One or more embodiments of the present disclosure relate to an insect repellent composition comprising: medium-chain triglycerides and a C12 fatty acid.

One or more embodiment of the present disclosure relate to insect repellent composition comprising; a hydrophobic phase and a hydrophilic phase, wherein the hydrophobic phase consists of medium-chain triglycerides and a C12 fatty acid, and wherein the hydrophilic phase creates a water-in-oil or oil-in-water emulsion.

### BRIEF DESCRIPTION OF THE FIGURES

The drawings and detailed description that follow are intended to be merely illustrative and are not intended to limit the scope of the present disclosure as contemplated. The detailed dclescription of specific embodiments of the present disclosure can be best understood when read in conjunction with the following drawings.

FIG. 1 is a graph showing the efficacy of C12 fatty acid in the biting fly blood feeding assay; and

FIG. 2 is a chart showing the efficacy of C12 fatty acid composition for repelling ticks as a function of time.

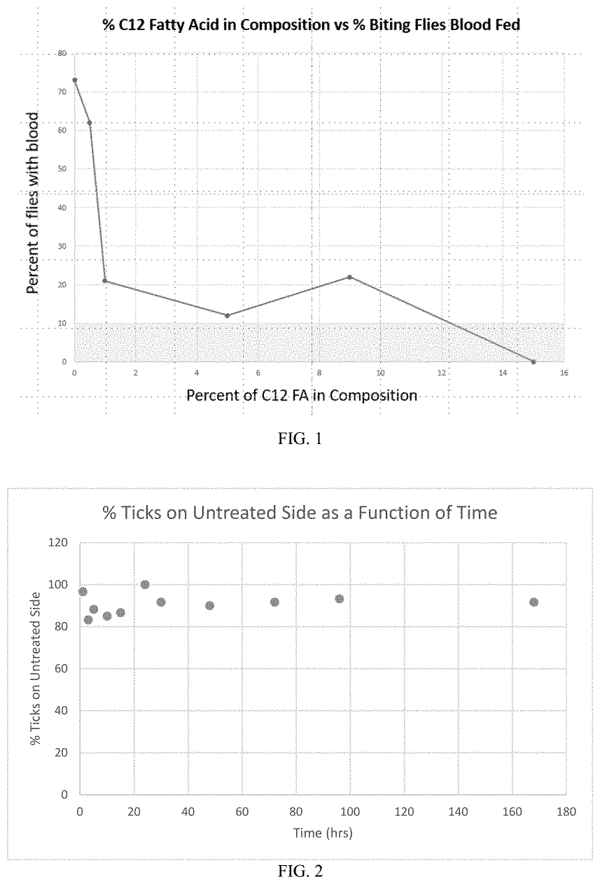

### DETAILED DESCRIPTION

Embodiments described herein describe a product composition that overcomes one or more of the aforementioned shortcomings of the prior art. While the disclosed applications of the present disclosure satisfy a long-felt but unmet need for an insect repellent with improved safety, efficacy, and aesthetics, it should be understood that the present disclosure is not limited to being implemented in the precise manners set forth herein, but could be implemented in other manners without undue experimentation by those of ordinary skill in the art in light of this disclosure. Accordingly, the examples set forth herein should be understood as being illustrative only and should not be treated as limiting.

As used herein, “natural” or “natural-derived” means that at least 70% of the composition is of natural origin. For instance, isopropyl myristate has a molecular weight of 270.45 g/mole. Isopropyl alcohol which has a molecular weight of 60.10 g/mole is likely made from a synthetic source while the myristic acid portion of the molecule is commonly sourced from either coconut or palm kernel oil. Therefore, the portion of the molecule that is synthetic is about 22% and the portion of the molecule that is natural or natural-derived is about 78%. The whole composition would be an average of the natural or natural-derived average of all molecules in the composition on a weight basis. Embodiments of the present disclosure will comprise, on average, over 70% by weight percent natural or natural-derived ingredients. If the formula is a lotion, the water fraction would not be considered in the calculation even though it is considered a natural material because it is simply being used as a carrier or diluent. Similarly, natural occurring minerals such as zinc oxide would also not be used as part of the calculation to determine “natural” content.

Prior art technologies of putting fatty acid and solvents in water and forming an emulsion to spray on skin result in a) skin irritation, b) drying of skin, or c) nasal irritation. The use of only even-chained C10 and C12 fatty acids should lead to an improved safety and aesthetic profile of product.

The present disclosure recognizes the issues discussed in the Background section above in order to develop skin-care products with the added benefit of insect repellency. Initial compositions included a mixture of essential oils, light-cut fatty acids (C8 and C10 fatty acid) and/or mid-cut fatty acids (C12 and C14 fatty acid), and medium-chain triglycerides (“MCT”—C8 and C10 alkyl chain lengths) in a homogeneous oil matrix for use as an insect repellent. To note, the terms light-cut and mid-cut fatty acids refer to distillation processes and their origins are from the chemical industry while the term medium-chain triglycerides originate from the food industry. The different origins may cause confusion to people unfamiliar with the different materials, their structural differences, origins and uses. Regardless, these compositions still had negatives when tested, including skin feel, odor, and cost. Further development led away from the use of C8 and C10 fatty acids. This approach led to improvements in skin feel, odor, and cost. However, an unexpected problem arose with these changes. While C12 fatty acid is reasonably soluble in medium-chain triglycerides, upon addition of essential oils and increased concentrations of C12 fatty acid, the C12 fatty acid separated from the mixture and subsequently precipitated from solution as C12 fatty acid crystals. Not to be bound by theory, this was believed to be caused by the smaller, bulkier-shaped essential oil components disrupting the lipid bilayer structure that naturally occurs when triglycerides are in a homogeneous solution.

The present disclosure therefore recognizes that a better matrix for both the essential oils and short-to-mid-chained fatty acid would be one that had less rigid or tightly packed alkyl chains associated with the lipid bilayer. This led to the idea of including a co-solvent with the mid-cut triglyceride (“MCT”) solvent, which would increase the MCT solvent's ability to solubilize both the essential oils and the C12 fatty acid within a single oil phase. Adding co-solvents such as oleochemicals containing a medium-length alkyl chains (C8 to C14) and a polar head group appear to solve the C12 fatty acid precipitation problem. In one or more embodiments, use of the term MCT means a triglyceride composed of a glycerin backbone with three ester linkages to three carboxylic acids primarily of C8 and C10 alkyl chain lengths. The polar head groups comprise methyl esters, isopropyl esters, fatty acids, alcohols, ketones, aldehydes, lactones, isethionates, and the like. The present disclosure is directed to a combination of skin-friendly and preferably natural or natural-derived ingredients that overcome one or more of the aforementioned shortcomings of the prior art. The present disclosure comprises a natural or natural-derived composition based on four main ingredients: essential oils, C12 fatty acid, mid-chain triglycerides, and a co-solvent in a homogeneous phase that meets all of the consumer desires of safety, efficacy, and aesthetics described above.

Co-solvents that are both natural-derived and help maintain a homogeneous phase include, for example: methyl, ethyl, propyl, isopropyl, butyl isobutyl, ethylhexyl, or isoamyl esters of capric, lauric, myristic, palmitic, oleic acids, and combinations thereof. Not all these esters are deemed safe for cosmetic or skin care applications, so each ester would need to be evaluated independently for safety and aesthetic properties. The same safety and aesthetic concerns are also important for the fatty acids, alcohols, ketones, aldehydes, lactones, isethionates, and the like. It may be possible to use lower amounts of these materials to maintain a stable single-phase system. It is also possible to odor strip these materials via vacuum distillation, counter-current steam distillation, air or nitrogen sparging, or passing the material through an absorbent bed containing nitrogen, polystyrene resin, clay, hydrogenation over catalyst, etc. to remove odor bodies prior to use. However, all must meet the same safety and aesthetic expectations as the other ingredients.

The MCT as a dilutant at high enough percentage by weight in the composition prevents undesirable crystallization down to about 45-50° F. (with only C10 fatty acid, the composition is stable to about 12% concentration in the formula and with only C12 fatty acid, the composition is stable up to about 7%). It was also determined that the combination of either C8 or C10 with the C12 in a ratio of 1:4 or greater results in no undesirable crystallization.

Both these findings indicated that stearic manipulations are very important. It was also determined that other types of co-solvents also limit crystallization. For instance, methyl decanoate and isopropyl myristate also limit crystallization when added to the composition. A 100% substitution of the MCT with these yielded a clear product at cooler temperatures. A 25% substitution level gave significant benefit but not to the total clarity. Other co-solvents recognized by the present disclosure as useful in preventing crystallization of fatty acids from the composition include alcohols such as methanol, ethanol, propanol, and isopropanol. However, as indicated earlier, these alcohols are drying to the skin and should be avoided, if at all possible, in skin-care type repellent formulas.

Other co-solvents that the present disclosure recognize as useful in preventing precipitation of fatty acids from the present formula include but are not limited to; Hexanediol CG, Phenylpropanol EHG, sorbitan laurate, polyglyceryl-4 laurate, dilauryl citrate, butylene glycol, cococaprylate caprate, gyclohexasiloxane, cyclopentasiloxane, ethoxy diglycol, FSS Supersolve (Alkylpolypentoside), Tecosolve 61 (Polyglyceryl-6 Caprylate; Polyglyceryl-4 Caprate; Polyglyceryl-4 Cocoate; Polyglyceryl-6 Ricinoleate), 1,3-propanediol, C12-C15 alkyl benzoate, dimethyl isosorbide, isododecane. From a consumer perspective, these co-solvents would be less desirable as they would be perceived by consumers as synthetic and not natural in origin.

Adjuncts that are desirable for skin health benefits include tocopherols, aloe gel, glycerin, titanium dioxide, zinc oxide, kaolin clay, cetearyl, cetyl A, coconut oil, olive oil, allantoin, aluminum hydroxide gel, colloidal oatmeal, monoglycerides, diglycerides, topical starch, zinc acetate, and zinc oxide. These same adjuncts would also be desirable in other product forms such as moisturizing lotions, moisturizing creams, moisturizing gels, shea butters, shampoos, conditioners, body washes, cleansing bars, etc.

It may also be desirable to add emollients which are known skin-conditioning agents. Fiume et al., International Journal of Cosmetics, Sep. 11, 2015, published an article titled “Safety Assessment of Alkyl Esters as Used in Cosmetics” which listed a variety of emollients that were determined to be safe on skin. The lists from this publication are incorporated by reference to include emollients the present disclosure considers useful as potential emollients.

Examples of oil-based formulas which fit within the present disclosure are shown below in Table 1. In order to make these formulas completely soluble, phase stable, skin friendly, and consumer perceived as natural, these formulas should be free of solvents such as silicones, mineral oils, petrolatum, alcohols, and other ingredients that are rated as greater than a 2 or 3 on the Environmental Working Group's website. Further, these formulas are free of fatty acids with a chain length shorter than 10, odd-numbered chain lengths, alkyl chains containing iso-branching, or their associated esters.

TABLE 1
Oil-Based Formula Examples

| Ingredient List | Formula 1 | Formula 2 | Formula 3 | Formula 4 | Formula 5 | Formula 6 | Formula 7 |
|---|---|---|---|---|---|---|---|
| C10 Fatty Acid | | 7.5% | 7.5% | 3% | 2.5% | 5% | |
| C12 Fatty Acid | 15% | 7.5% | 7.5% | 12% | 10% | 10% | 15% |
| Isopropyl Myristate | 10% | | | 5% | 7.5% | | |
| Methyl Laurate | | | | | | 5% | 10% |
| Medium Chain | 70% | 80% | 80% | 75% | 75% | 75% | 70% |
| Triglycerides | | | | | | | |
| Essential Oil | 5% | 5% | | 5% | | 5% | |
| Mixture 1 | | | | | | | |
| Essential Oil | | | 5% | | 5% | | 5% |
| Mixture 2 | | | | | | | |

Essential Oil Mixture 1 contains a mixture of essential oils including oils from at least 4 of the following materials; lavender, lemongrass, rosemary, cedarwood, chamomile, eucalyptus, geranium, ylang, bergamot, rosemary, citronella, peppermint, patchouli, neem, and rose geranium.

Essential Oil Mixture 2 contains a mixture of essential oils, including at least 3 oils from the following materials; lemongrass, citronella, peppermint, eucalyptus chamomile, geranium, and rosemary. Further, the Essential Oil Mixture 2 is free of lavender oil, cedarwood oil, and neem oil.

The formulas contained in Table 1 may be dispensed using a misting sprayer, aerosol can, roller ball, nonwoven fabric wipe, or drop-wise dispensing lid type container package.

Examples of emulsion-based lotion formulas which fit within the present disclosure are shown below in Table 2. In order to make these formulas completely soluble, phase stable, skin friendly, and consumer perceived as natural, these formulas are free of solvents such as silicones, mineral oil, petrolatum, alcohols, and other ingredients that are rated as greater than a 2 or 3 on the Environmental Working Group's website. Further, these formulas are essentially free of fatty acids (below 0.1%) of a chain-length shorter than 10, odd-numbered chain-lengths, alkyl chains containing iso-branching, or their associated esters.

TABLE 2
Examples of Emulsion-Type Repellent Lotion Formulas.

| | Formula | Formula | Formula | Formula | Formula |
|---|---|---|---|---|---|
| Ingredients | 1 | 2 | 3 | 4 | 5 |
| Water phase | | | | | |
| Water | 56.00% | 60.00% | 54.00% | 53.00% | 50.00% |
| Sclerotium Gum | 0.50% | 0.60% | 0.40% | 0.40% | 0.30% |
| Glycerin | 5.00% | 5.50% | 4.00% | 4.00% | 3.75% |
| Tetrasodium | 0.10% | 0.10% | 0.10% | 0.10% | 0.90% |
| Glutamate, | | | | | |
| Diacetate | | | | | |
| Propanediol | 0.50% | 0.60% | 0.50% | 0.50% | 0.45% |
| Allantoin | 0.50% | 0.60% | 0.50% | 0.50% | 0.45% |
| Aloe Vera Gel | 1.20% | 1.20% | 1.00% | 1.00% | 0.80% |
| Oil Phase | | | | | |
| C10 Fatty Acid | 5.00% | 3.00% | 7.00% | 7.50% | 5.00% |
| C12 Fatty Acid | 10.00% | 6.00% | 7.00% | 7.50% | 10.00% |
| Isopropyl | | 3.00% | 3.50% | 3.00% | 3.00% |
| Myristate | | | | | |
| Olive Oil | 2.00% | 1.00% | 1.00% | 1.00% | 1.00% |
| Medium Chain | 2.00% | 1.00% | 0.50% | 0.50% | 2.00% |
| Triglycerides | | | | | |
| Dimethicone | | 0.25% | 0.50% | 0.50% | 0.82% |
| Meadowfoam | 0.50% | 0.30% | 0.50% | 0.50% | 0.75% |
| BTMS16 | 0.60% | 0.40% | 0.60% | 0.60% | 0.75% |
| Emulsifying | 4.00% | 4.50% | 5.00% | 5.00% | 5.75% |
| Wax | | | | | |
| Shea Butter | 3.50% | 3.00% | 3.50% | 3.50% | 4.00% |
| Stearic Acid | 1.00% | 1.20% | 1.50% | 1.50% | 1.75% |
| Cetyl Alcohol | 1.00% | 1.00% | 1.17% | 1.20% | 1.50% |
| Cetearyl Alcohol | 0.60% | 0.60% | 0.70% | 0.70% | 0.80% |
| Vitamin E | 0.80% | 0.75% | 1.00% | 1.00% | 1.00% |
| Cyclomethicone | 1.50% | 1.60% | 1.70% | 1.70% | 1.90% |
| Cosmocil CQ | 1.00% | 1.00% | 1.00% | 1.00% | 1.00% |
| Citric Acid | 0.15% | 0.15% | 0.15% | 0.15% | 0.15% |
| Ascorbic Acid | 0.18% | 0.15% | 0.18% | 0.15% | 0.18% |
| Fragrance | | | | | |
| Essential Oil | | 2.50% | 3.00% | | 2.00% |
| Mixture 1 | | | | | |
| Essential Oil | 2.37% | | | 3.50% | |
| Mixture 2 | | | | | |

While small amounts of synthetic materials, such as dimethicone, may be necessary to stabilize the emulsion or to provide a high-quality skin feel, these materials should be kept to as low as possible, such as below 1%.

Examples of butter cream-type repellent formulas which fit within the present disclosure are shown below in Table 3. In order to make these formulas completely soluble, phase stable, skin friendly, and consumer perceived as natural, these formulas are free of solvents such as silicones, mineral oil, petrolatum, alcohols, and other ingredients that are rated as greater than a 2 or 3 on the Environmental Working Group's website. Further, these formulas are essentially free of fatty acids (below 0.1%) of a chain-length shorter than 10, odd-numbered chain-lengths, alkyl chains containing iso-branching, or their associated esters.

TABLE 3
Examples of Butter Cream-Type Repellent Formulas.

| | Formula | Formula | Formula | Formula | Formula |
|---|---|---|---|---|---|
| Ingredients | 1 | 2 | 3 | 4 | 5 |
| C10 Fatty Acid | 3.00% | 5.00% | 7.50% | 7.50% | 10.00% |
| C12 Fatty Acid | 12.00% | 5.00% | 7.50% | 7.50% | 10.00% |
| Isopropyl Myristate | 1.00% | | | 1.00% | |
| Methyl Laurate | 1.00% | 1.00% | | | |
| Medium Chain | 5.00% | 1.00% | | 1.50% | 1.00% |
| Triglycerides | | | | | |
| Olive Oil | 20.00% | 26.00% | 28.50% | 25.00% | 25.00% |
| Shea Butter | 56.50% | 60.00% | 54.00% | 55.00% | 51.00% |
| Vitamin E | 1.00% | 1.00% | 1.00% | 1.00% | 1.00% |
| Essential Oil Mix 1 | 0.50% | | 1.50% | | 2.00% |
| Essential Oil Mix 2 | | 1.00% | | 1.50% | |

Liquid cleaning formulas, which would be used as body or facial washes, are also embodiments of the present disclosure, wherein the amounts of C10 fatty acid, C12 fatty acid, and MCT were used in the amounts of 1.5%, 1.5%, and 1.0%, respectively. This type of product would be of value for people that seem to be bothered around the face, eyes and ears, but not with the rest of their bodies.

Bar-soap type formulas are also embodiments of the present disclosure. The key with making bar soaps is to make sure the soap melt is pre-made and not over-saponified (extra residual caustic in the soap). Then a mixture of C10, C12, and MCT (between 0.5 and 4%) can be added to the melt at the very end of processing with minimal mixing. A range of each ingredient, between 0.3% and 1.5% and 0.1% and 1.0% by weight, respectively, would be added.

It should be noted that a sunscreen benefit may be added to each of these formulas using either natural inorganic sunscreen ingredients such as zinc oxide or titanium dioxide, or natural organic sunscreen ingredients, which could include but are not limited to the following: carrot seed oil, red raspberry seed oil, almond oil, coconut oil, turmeric, vitamin E, and aloe vera. Other synthetic organic ingredients are also contemplated. These ingredients include but are not limited to avobenzone, oxybenzone, homosalate, octisalate, octocrylene, and octinoxate.

Specific liquid emollients contemplated by the present disclosure include but are not limited to: C12-15 alkyl benzoate, octyldodecanol, isopropyl palmitate, canola oil, shea butter ethyl esters, and dicaprylyl carbonate.

It may also be desirable to use only ingredients that have been evaluated by the Environmental Working Group as having a Hazard Score of less than 3, specifically a score of 1 or 2 on their scale of 1 to 10. This low score reinforces the safety profile of personal care and cosmetic products for use on human skin.

In one embodiment of the present disclosure, a natural-derived ingredient insect-repellent personal-care composition comprises a hydrophobic solvent system containing medium-chain triglycerides and a co-solvent wherein the co-solvent is selected from the group comprising: an alkyl methyl ester, alkyl ethyl ester, alkyl propyl ester, alkyl isopropyl ester, an alkyl carboxylic acid, an alkyl isethionate, an alkyl ketone, and combination thereof, wherein the alkyl chain contains between ten and fourteen carbon atoms. In another embodiment of a natural-derived ingredient insect-repellent composition, the hydrophobic solvent system is part of a single-phase, dual-phase, or triple-phase personal-care product composition. A single-phase system would be an oil phase system, a dual-phase system would consist of water and oil phases, and a triple-phase system would consist of an oil, water, and solid phase, such as when something like zinc oxide or titanium oxide is added to a dual-phase system, or when a small amount of oil phase is added to a solid soap formula containing saponified fat and water in a solid emulsion system.

In another embodiment of the present disclosure, a natural-derived ingredient insect-repellent composition, the concentration of the medium-chain triglyceride in the hydrophobic phase is between 30% and 95%, in other embodiments between 40% and 90%, in other embodiments between 50% and 85%, and in yet other embodiments between 60% and 80%. In another embodiment, the concentration of the medium-chain triglyceride in a butter-cream formula is between 0.5% and 10%, in other embodiments between 1% and 5%, and in yet other embodiment between 1.5% and 3%.

In another embodiment of the present disclosure, a natural-derived ingredient insect-repellent composition, the ratio of the medium-chain triglyceride to an alkyl methyl ester or alkyl isopropyl ester in a lotion, butter cream, cleanser, or solid soap formula is between 1:2 and 4:1, in other embodiments between 1:1.5 and 3:1, and in yet other embodiments between 1:1 and 2:1.

In another embodiment of the present disclosure, a natural-derived ingredient insect-repellent composition also comprises: one or more fatty acids containing from 8 to 12 carbons, and wherein the C12 fatty acid represents at least 50% of the total fatty acid composition, in other embodiments at least 60% of the total fatty acid composition, in other embodiments at least 70% of the total fatty acid composition, in other embodiments at least 80% of the total fatty acid composition, in other embodiments at least 90% of the total fatty acid composition, in other embodiments at least 95% of the total fatty acid composition, and in yet other embodiments at least 98% of the total fatty acid composition.

In another embodiment of the present disclosure, a natural-derived ingredient insect-repellent composition also comprises: one or more fatty acids containing from 8 to 12 carbons, wherein the C12 fatty acid comprises at least 50% of the total fatty acid composition and wherein the C8 fatty acid comprises less than 30% of the total fatty acid composition, in other embodiments less than 20% of the total fatty acid composition, in other embodiments less than 10% of the total fatty acid composition, in other embodiments less than 5% of the total fatty acid composition, in other embodiments less than 2% of the total fatty acid composition, and in yet other embodiments less than 1% of the total fatty acid composition.

In another embodiment of the present disclosure, the total fatty acid concentration in the natural-derived ingredient insect-repellent composition is at least 4.5% by weight, in other embodiments at least 7.5% by weight, in other embodiments at least 10% by weight, in other embodiments at least 12.5% by weight, in other embodiments at least 15% by weight, in other embodiments at least 20% by weight, in other embodiments at least 25% by weight, and in yet other embodiments at least 30% by weight.

In another embodiment of the present disclosure, the natural-derived ingredients insect-repellent composition includes a mixture of essential oils, wherein the mixture includes at least one essential oil selected from the group consisting of lavender oil, lemongrass oil, rosemary oil, cedarwood oil, and combinations thereof.

In another embodiment of the present disclosure, the natural-derived ingredients insect-repellent composition includes a mixture of essential oils, wherein the mixture includes at least one essential oil selected from the group of lemongrass oil, citronella oil, peppermint oil, eucalyptus oil, and combinations thereof.

In another embodiment of the present disclosure, the total essential oil concentration of the natural-derived ingredients insect-repellent composition represents at least 1% of the total composition by weight, in other embodiments at least 2% of the total composition by weight, in other embodiments 3% of the total composition by weight, in other embodiments 4% of the total composition by weight, in other embodiments 5% of the total composition by weight, in other embodiments 10% of the total composition by weight, in other embodiments 15% of the total composition by weight, and in yet other embodiments 20% of the total composition by weight.

In another embodiment of the present disclosure, the ratio of C10 fatty acid to C12 fatty acid is between 1:50 and 1:2, between 1:25 and 1:3, or between 1:20 and 1:4.

In another embodiment of the present disclosure, the ratio of C12 fatty acid to the co-solvent is between 1:4 and 4:1, in other embodiments between 1:3 and 3:1, in yet other embodiments between 1:2 and 2:1.

In another embodiment of the present disclosure, the ratio of essential oil to fatty acid is 1:30 to 1:2, in other embodiments between 1:15 and 1:3, in yet other embodiments between 1:10 and 1:4 on a weight basis.

In another embodiment of the present disclosure, the natural-derived ingredients insect-repellent composition is thermally stable to a temperature of 60° F., in other embodiments to a temperature of 55° F., an in yet other embodiments to a temperature of 50° F.

In another embodiment of the present disclosure, the tick repellent efficacy of the natural-derived ingredients insect-repellent composition is five days or greater as measured by the acaricidal activity test described by Barrozo.

In another embodiment of the present disclosure, the mosquito repellent efficacy of the natural-derived ingredients insect-repellent composition is three hours or greater as measured by the arm in cage test (Logan, J. G., Stanczyk, N. M., Hassanali, A. et al. Arm-in-cage testing of natural human-derived mosquito repellents. Malar J 9, 239 (2010). https://doi.org/10.1186/1475-2875-9-239).

In another embodiment of the present disclosure, the biting fly repellent efficacy of the natural-derived ingredients insect-repellent composition is at least twenty-four hours as measured using the biting fly blood feeding assay (Zhu J J, Cermak S C, Kenar J A, et al. Better than DEET Repellent Compounds Derived from Coconut Oil. Scientific Reports. 2018 Sep. 8 (1):14053. DOI: 10.1038/s41598-018-32373-7. PMID: 30232355; PMCID: PMC6145915).

In another embodiment of the present disclosure, the tick repellent efficacy is of the natural-derived ingredients insect-repellent composition at least five days as measured using the tick repellency bioassay method described by Barrozo.

In another embodiment of the present disclosure, the natural-derived ingredients insect-repellent composition contains medium-chain triglycerides and lauric acid, and optionally a range of hydrophobic carrier vegetable oils, plant-based waxes, or plant-based butters wherein the repellent composition can be applied directly to the skin by direct human application, a spray or spritz dispenser, a roller-ball, a non-woven wipe, or a drop-wise applicator. In another embodiment, the natural-derived ingredients insect-repellent composition contains a hydrophobic phase consisting of medium-chain triglycerides and lauric acid and a hydrophilic phase designed to create a water-in-oil or oil-in-water emulsion having the consistency of a lotion, cream, or butter.

In another embodiment of the present disclosure, the solvent and fatty acids of the natural-derived ingredients insect-repellent composition are derived from coconut oil or palm kernel oil.

In another embodiment of the present disclosure, the solvent and fatty acids of the natural-derived ingredients insect-repellent composition are dispensed using a wet-wipe type of consumer product.

In another embodiment of the present disclosure, the natural-derived ingredients insect-repellent composition is used in an array of skin-care products such as those selected from the group consisting of a single-phase oil skin-care product, a bi-continuous hydrophobic phase skin-care product, a skin-care lotion product, a skin-care cream product, a skin-care butter-cream product, a sun-tan lotion product, a sun-screen product, a skin-care liquid cleansing product, a skin-care cleansing bar product, a hair-care shampoo product, a hair-care conditioning product, a pet-care shampoo product, a pet-care hair oil product, and combinations thereof.

In another embodiment of the present disclosure, the average natural or natural-derived content of the ingredients of the natural-derived ingredients insect-repellent composition averages 70% or more on a weight basis.

It should be understood that the present disclosure may be combined with other pre-existing and yet-to-be invented natural-derived skin care product compositions. For example, such as moisturizing lotions, moisturizing creams, moisturizing gels, shampoos, conditioners, etc. While specific embodiments of the present disclosure were illustrated and described herein, variations and modifications may be made by those skilled in the art without departing from the scope of this disclosure. The present disclosure is for purposes of illustration and not of limitation; it may take many forms other than those explicitly disclosed herein. As such, the claims below shall be read to include all obvious variations and modifications that may be within the spirit of the present disclosure.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In various embodiments disclosed herein, a single component can be replaced by multiple components and multiple components can be replaced by a single component to perform a given function or functions. Except where such substitution would not be operative, such substitution is within the intended scope of the embodiments. The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended that the scope of the present disclosure to be defined by the claims appended hereto. As such, the claims below shall be read to include all obvious variations and modifications that may be within the spirit of the present disclosure.

What is claimed is:

1. An insect repellent composition comprising: medium-chain triglycerides and a co-solvent, wherein the co-solvent comprises an alkyl chain comprising between eight and fourteen carbon atoms.

2. The insect repellent composition of claim 1 wherein the co-solvent is selected from a group consisting of an alkyl methyl ester, an alkyl ethyl ester, an alkyl isopropyl ester, an alkyl carboxylic acid, an alkyl methyl ketone, an alkyl ethyl ketone, an alkyl alcohol, an alkyl isethionate, and combinations thereof

3. The insect repellent composition of claim 1 wherein the concentration of the medium-chain triglyceride in the composition is between 50 and 85% by weight of the total insect repellent composition.

4. The insect repellent composition of claim 1 wherein the composition further comprises one or more fatty acids containing from 8 to 12 carbons, wherein one of the one or more fatty acids is a C12 fatty acid, and wherein the C12 fatty acid represents at least 50% of a total fatty acid composition by weight.

5. The insect repellent composition of claim 1 wherein the composition further comprises two or more fatty acids containing from 8 to 12 carbons, wherein one of the two or more fatty acids is a C12 fatty acid and wherein one of the two or more fatty acids is a C8 fatty acid, wherein the C12 fatty acid comprises at least 50% of a total fatty acid composition, and wherein the C8 fatty acid comprises less than 2% of the total fatty acid composition.

6. The insect repellent composition of claim 4 wherein the total fatty acid concentration in the composition is between 7.5% and 25% by weight.

7. The ingredient insect repellent composition of claim 4 further comprising a mixture of essential oils wherein the mixture of essential oils includes at least one essential oil selected from the group consisting of lavender oil, lemongrass oil, rosemary oil, cedarwood oil, and combinations thereof; and wherein the essential oil mixture concentration is between 3% and 10% of the total insect repellent composition on a weight basis.

8. The ingredient insect repellent composition of claim 4 wherein one of the one or more fatty acids is a C10 fatty acid, and wherein the ratio of the C10 fatty acid to the C12 fatty acid is between 1:50 and 1:2 on a weight basis.

9. The insect repellent composition of claim 4 wherein the insect repellent composition has a tick repellent efficacy of five days or greater, a mosquito repellent efficacy of at least 3 hours, and a biting fly repellent efficacy of twenty-four hours.

10. The ingredient insect repellent composition of claim 7 wherein the ratio of the C12 fatty acid to the co-solvent is between 1:2 and 2:1 on a weight basis; and wherein the ratio of the essential oil mixture to the one or more fatty acids is between 1:10 and 1:4 on a weight basis.

11. The insect repellent composition of claim 7 wherein the medium-chain triglycerides, the co-solvent, the mixture of essential oils, and the one or more fatty acids are each derived from naturally occurring plant-based materials.

12. The insect repellent composition of claim 11 wherein the medium-chain triglycerides, the co-solvent, and the one or more fatty acids are derived from coconut oil or palm kernel oil.

13. The insect repellent composition of claim 7 wherein the insect repellent composition is thermally stable at a temperature of 50° F.

14. The insect repellent composition of claim 1 wherein the medium-chain triglycerides and the co-solvent have an average natural or natural-derived content of at least 70%.

15. An insect repellent composition comprising medium-chain triglycerides and a C12 fatty acid.

16. The insect repellent composition of claim 15 further comprising a hydrophobic co-solvent, wherein the hydrophobic co-solvent comprises an alkyl chain comprising between eight and fourteen carbon atoms.

17. The insect repellent composition of claim 16, further comprising at least one of carrier vegetable oils, plant-based waxes, plant-based butters, and combinations thereof

18. The insect repellent composition of claim 15 wherein the insect repellent composition is directly applicable to mammalian skin and wherein the insect repellent composition is directly applicable through a delivery mechanism selected from the group consisting of direct application, application by a spray or spritz dispenser, application by a rollerball, application by a non-woven wipe, application by a drop-wise dispensing flexible package applicator, and combinations thereof.

19. An insect repellent composition comprising; a hydrophobic phase and a hydrophilic phase, wherein the hydrophobic phase consists of medium-chain triglycerides and a C12 fatty acid, and wherein the hydrophilic phase and hydrophobic phase together creates an water-in-oil emulsion or an oil-in-water emulsion.

20. The insect repellent composition to claim 19 further comprising at least one natural sunscreen component, and wherein the natural sunscreen component is selected from the group consisting of zinc oxide, titanium dioxide, carrot seed oil, red raspberry seed oil, almond oil, coconut oil, turmeric, vitamin E, aloe Vera, and combinations thereof.
